(19) United States
(12) Patent Application Publication
(10) Pub. No.: US 2021/0287765 A1
(43) Pub. Date: Sep. 16, 2021

(54) SYSTEMS AND METHODS FOR GENERATING AND SEARCHING A CHEMICAL COMPOUND DATABASE

(71) Applicant: Collaborative Drug Discovery, Inc., Burlingame, CA (US)

(72) Inventors: Barry A. BUNIN, Belmont, CA (US); Peter GEDECK, Arlington, VA (US)

(73) Assignee: Collaborative Drug Discovery, Inc., Burlingame, CA (US)

(21) Appl. No.: 17/196,420

(22) Filed: Mar. 9, 2021

Related U.S. Application Data
(60) Provisional application No. 62/989,008, filed on Mar. 13, 2020.

(51) Int. Cl. G16C 20/80; G16C 20/90; G16C 20/70; G16C 20/20; G06F 16/901

(52) U.S. Cl. CPC G16C 20/80; G16C 20/90; G06F 16/9024; G16C 20/20; G16C 20/70

(57) ABSTRACT

A method includes identifying, using one or more processors, a first plurality of fragments of a first structure graph representing a first chemical compound, and generating, using the one or more processors, a first plurality of subgraphs of the first structure graph based on the first plurality of fragments. The method includes generating, using the one or more processors, a first plurality of nodes based on the first plurality of subgraphs. The method includes arranging, using the one or more processors, the first plurality of nodes based on a number of the first plurality of fragments associated with each of the first plurality of subgraphs. The method includes connecting, using the one or more processors, the first plurality of nodes using a first plurality of edges and based on one or more reduced graph rules.

### CROSS-REFERENCE TO RELATED APPLICATIONS

This application claims the benefit of and priority to U.S. Provisional Application No. 62/989,008 filed on Mar. 13, 2020. The disclosure of the above application is incorporated herein by reference.

### GOVERNMENT LICENSE RIGHTS

This invention was made with government support under TR002527 awarded by the National Institutes of Health. The government has certain rights in the invention. 37 CFR 401.14(f)(4).

### FIELD

The present disclosure relates to systems and methods for generating and searching a chemical compound database.

### BACKGROUND

The statements in this section merely provide background information related to the present disclosure and may not constitute prior art.

When developing a new drug, a medicinal chemist may identify a chemical compound to advance into animal studies and human clinical trials. To identify the chemical compound, medicinal chemists may start with a set of lead compounds that demonstrate efficacy in achieving a desired biological effect and modify the lead compounds to achieve a desired level of potency and other pharmacological properties (e.g., absorption, distribution, metabolism, excretion, toxicity, among others). To identify the modified compounds, a medicinal chemist may divide molecules of the lead compounds into their constituent fragments, compare the structures to a different lead compound due to substitution of fragments, and review associated experimental data to evaluate how the presence or absence of the constituent fragments relates to the pharmacological properties. As such, medicinal chemists may analyze numerous variations of the lead compounds to achieve both a desired level of potency and other pharmacological properties, thereby making the identification of the chemical compound a time-consuming process.

Furthermore, when evaluating modifications to an identified lead compound, medicinal chemists may determine whether further exploration of a given modification is feasible based on structure-activity relationship (SAR) data, such as similarity (i.e., substituting similar fragments should yield similar activity), additivity (i.e., contributions of substituents to activity are independent from each other), non-additivity, Free-Wilson analysis, among others. To generate the SAR data, the medicinal chemist may manually define, using a computing system, the core structures and fragments, thereby causing the computing system to generate the SAR data. However, verifying and characterizing the SAR data is a time-consuming process that may require substantial computing resources.

### SUMMARY

This section provides a general summary of the disclosure and is not a comprehensive disclosure of its full scope or all of its features.

The present disclosure provides a method for generating a chemical compound graph database including identifying, using one or more processors configured to execute instructions stored in a nontransitory computer-readable medium, a first plurality of fragments of a first structure graph representing a first chemical compound. The method includes generating, using the one or more processors, a first plurality of subgraphs of the first structure graph based on the first plurality of fragments. The method includes generating, using the one or more processors, a first plurality of nodes based on the first plurality of subgraphs, where each node of the first plurality of nodes corresponds to a respective subgraph of the first plurality of subgraphs. The method includes arranging, using the one or more processors, the first plurality of nodes based on a number of the first plurality of fragments associated with each of the first plurality of subgraphs. The method includes connecting, using the one or more processors, the first plurality of nodes using a first plurality of edges and based on one or more reduced graph rules.

In some forms, the method further includes identifying, using the one or more processors, a second plurality of fragments of a second structure graph representing a second chemical compound. The method further includes generating, using the one or more processors, a second plurality of subgraphs of the second structure graph based on the second plurality of fragments. The method further includes generating, using the one or more processors, a second plurality of nodes based on the second plurality of subgraphs, where each node of the second plurality of nodes corresponds to a respective subgraph of the second plurality of subgraphs. The method includes arranging, using the one or more processors, the second plurality of nodes based on a number of the second plurality of fragments associated with each of the second plurality of subgraphs. The method includes connecting, using the one or more processors, the second plurality of nodes using a second plurality of edges and based on the one or more reduced graph rules.

In some forms, the method further includes identifying, using the one or more processors, one or more shared nodes from among the first plurality of nodes and the second plurality of nodes and merging, using the one or more processors, the first plurality of nodes and the second plurality of nodes at the one or more shared nodes.

In some forms, the method further includes generating one or more data entries of the chemical compound graph database based on the merged first plurality of nodes and the second plurality of nodes.

In some forms, each fragment of the first plurality of fragments is linked to a ring molecule of the first chemical compound.

In some forms, the one or more reduced graph rules further comprises connecting the first plurality of nodes using the first plurality of edges based on a nontransitive reduction routine.

In some forms, the one or more reduced graph rules further comprises connecting the first plurality of nodes using the first plurality of edges to form a Hasse diagram.

The present disclosure provides a system for generating a chemical compound graph database including one or more processors and a nontransitory computer-readable medium comprising instructions that are executable by the one or more processors. The instructions include identifying a first plurality of fragments of a first structure graph representing a first chemical compound. The instructions include generating a first plurality of subgraphs of the first structure graph based on the first plurality of fragments. The instructions include generating a first plurality of nodes based on the first plurality of subgraphs, where each node of the first plurality of nodes corresponds to a respective subgraph of the first plurality of subgraphs. The instructions include arranging the first plurality of nodes based on a number of the first plurality of fragments associated with each of the first plurality of subgraphs. The instructions include connecting the first plurality of nodes using a first plurality of edges and based on one or more reduced graph rules.

In some forms, the instructions further include identifying, using the one or more processors, a second plurality of fragments of a second structure graph representing a second chemical compound. The instructions further include generating, using the one or more processors, a second plurality of subgraphs of the second structure graph based on the second plurality of fragments. The instructions further include generating, using the one or more processors, a second plurality of nodes based on the second plurality of subgraphs, where each node of the second plurality of nodes corresponds to a respective subgraph of the second plurality of subgraphs. The instructions further include arranging, using the one or more processors, the second plurality of nodes based on a number of the second plurality of fragments associated with each of the second plurality of subgraphs. The instructions further include connecting, using the one or more processors, the second plurality of nodes using a second plurality of edges and based on the one or more reduced graph rules.

In some forms, the instructions further include identifying, using the one or more processors, one or more shared nodes from among the first plurality of nodes and the second plurality of nodes and merging, using the one or more processors, the first plurality of nodes and the second plurality of nodes at the one or more shared nodes.

In some forms, the instructions further include generating one or more data entries of the chemical compound graph database based on the merged first plurality of nodes and the second plurality of nodes.

In some forms, each fragment of the first plurality of fragments is linked to a ring molecule of the first chemical compound.

In some forms, the one or more reduced graph rules further comprises connecting the first plurality of nodes using the first plurality of edges based on a nontransitive reduction routine.

In some forms, the one or more reduced graph rules further comprises connecting the first plurality of nodes using the first plurality of edges to form a Hasse diagram.

The present disclosure provides a method including identifying, using one or more processors configured to execute instructions stored in a nontransitory computer-readable medium, a node from among a plurality of nodes stored in the chemical compound graph database based on an input received by the one or more processors, where the node corresponds to one or more fragments of a chemical compound. The method includes identifying, using the one or more processors, one or more related nodes from among the plurality of nodes associated with the node based on one or more structure-activity relationship rules. The method includes generating, using the one or processors, a structure activity relationship analysis based on the one or more related nodes and the node.

In some forms, the plurality of nodes form a Hasse diagram.

In some forms, the one or more structure-activity relationship rules comprise identifying, as the one or more related nodes, one or more child nodes associated with the node, one or more grandchildren nodes associated with the node, one or more parent nodes, one or more grandparent nodes, or a combination thereof.

In some forms, the structure activity relationship analysis includes a Free-Wilson analysis, an additivity analysis, a non-additivity analysis, or a combination thereof.

In some forms, the plurality of nodes include a first plurality of nodes and a second plurality of nodes, the first plurality of nodes represent a first chemical compound, and the second plurality of nodes represent a second chemical compound. In some forms, the first plurality of nodes are connected by a first plurality of edges and based on a nontransitive reduction routine, and the second plurality of nodes are connected by a second plurality of edges and based on the nontransitive reduction routine.

In some forms, the first plurality of nodes and the second plurality of nodes are merged at one or more shared nodes from among the first plurality of nodes and the second plurality of nodes.

Further areas of applicability will become apparent from the description provided herein. It should be understood that the description and specific examples are intended for purposes of illustration only and are not intended to limit the scope of the present disclosure.

### DRAWINGS

In order that the disclosure may be well understood, there will now be described various forms thereof, given by way of example, reference being made to the accompanying drawings, in which:

FIG. 1 illustrates a functional block diagram of a chemical compound system and a user device in accordance with the teachings of the present disclosure;

FIG. 2 illustrates a skeletal formula of a chemical compound in accordance with the teachings of the present disclosure;

FIG. 3 illustrates one or more identified fragments of a chemical compound in accordance with the teachings of the present disclosure;

FIG. 4 illustrates one or more subgraphs of a chemical compound in accordance with the teachings of the present disclosure;

FIG. 5A illustrates a plurality of nodes in accordance with the teachings of the present disclosure;

FIG. 5B illustrates a vertical arrangement of a plurality of nodes in accordance with the teachings of the present disclosure;

FIG. 5C illustrates a plurality of nodes connected based on one or more reduced graph rules in accordance with the teachings of the present disclosure;

FIG. 6 illustrates a plurality of chemical compounds having one or more shared nodes in accordance with the teachings of the present disclosure;

FIG. 7A illustrates one or more fragments that are identified based on one or more structure activity relationship rules in accordance with the teachings of the present disclosure;

FIG. 7B illustrates one or more fragments that are identified based on one or more structure activity relationship rules in accordance with the teachings of the present disclosure;

FIG. 8 is a flowchart of an example control routine in accordance with the teachings of the present disclosure; and

FIG. 9 is a flowchart of another example control routine in accordance with the teachings of the present disclosure.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

The present disclosure provides a computing system that includes a chemical compound graph database that includes graph structures for semantic queries. The graph structures include nodes, edges, and properties to represent various chemical compounds. More particularly, the chemical compound graph database links chemical compounds having structurally analogous fragments to form a SAR neighborhood, and the fragments are stored and arranged using a semilattice node structure, such as a Hasse diagram. As such, medicinal chemists may efficiently navigate among related chemical compounds to collate and analyze modifications to a chemical compound when identifying a chemical compound to advance into animal studies and human clinical trials. Furthermore, the chemical compound graph database enables a computing system to efficiently generate and provide the SAR data in reduced time and using reduced computational resources.

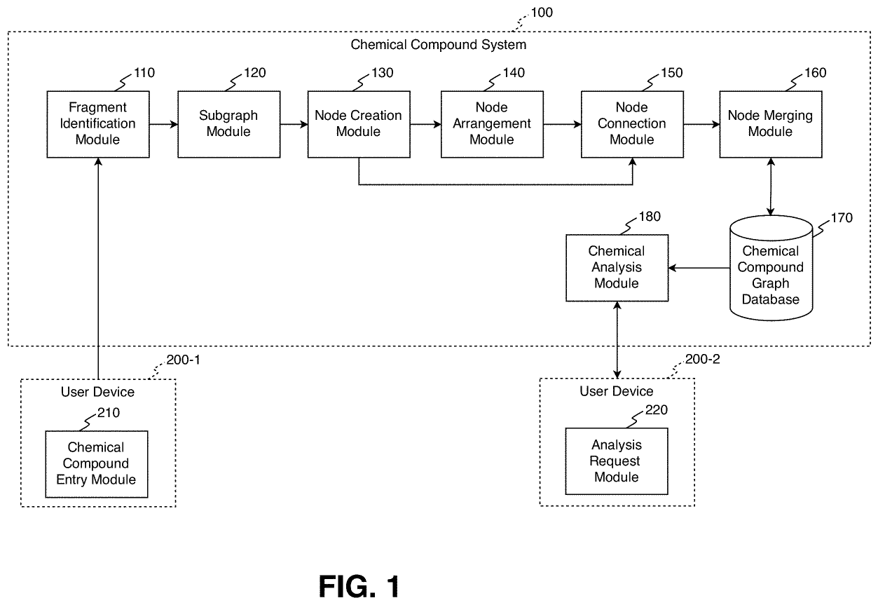

Referring to FIG. 1, a functional block diagram of a chemical compound system 100 and user devices 200-1, 200-2 (collectively referred to herein as user devices 200) is provided. In one form, the chemical compound system 100 and the user devices 200 are communicably coupled using a wired communication protocol and/or a wireless communication protocol (e.g., a Bluetooth®-type protocol, a cellular protocol, a wireless fidelity (Wi-Fi)-type protocol, a near-field communication (NFC) protocol, an ultra-wideband (UWB) protocol, among others).

In one form, the chemical compound system 100 is a computing system that includes one or more computing devices (e.g., one or more edge computing devices, multiple virtual computing devices including virtual computing resources, among others), one or more databases, among other computing system components. The chemical compound system 100 is configured to generate one or more database entries representing chemical compounds, fragments thereof, and/or relationships among the chemical compounds based on an input received from the user device 200-1, as described below in further detail. Furthermore, the chemical compound system 100 is configured to generate and provide a SAR analysis of one or more fragments associated with a given chemical compound to the user device 200-2 for display and manipulation, as described below in further detail.

In one form, the user devices 200 are computing devices including, but not limited to: a desktop computer, laptop, smartphone, tablet, personal digital assistant (PDA), and/or wearable device. It should be understood that the user devices 200 may be other suitable devices suitable for performing the functions described herein and are not limited to the examples described herein. Furthermore, while FIG. 1 illustrates two user devices 200, it should be understood that any number of user devices 200 may be included in other forms (e.g., one or more than two user devices 200).

The user devices 200-1 includes a chemical compound entry module 210, and the user device 200-2 includes an analysis request module 220. The chemical compound entry module 210 is configured to enable a user (e.g., a medicinal chemist or developer of the chemical compound system 100) to initiate generation of one or more database entries of the chemical compound system 100. Accordingly, in one form, the chemical compound entry module 210 is configured to provide one or more interface elements (e.g., audio instructions, graphical user interface, etc.) operable by the user to input information representing a given chemical compound.

The analysis request module 220 is configured to enable a user to navigate the chemical compound system 100 to identify one or more fragments associated with a given chemical compound, and to obtain, generate, and/or display a SAR analysis of one or more fragments associated with the given chemical compound. Accordingly, in one form, the analysis request module 220 is configured to provide one or more interface elements (e.g., audio instructions, graphical user interface, etc.) operable by the user to submit a request to the chemical compound system 100 and to provide the SAR analysis.

The chemical compound entry module 210 and/or the analysis request module 220 are configured to exchange information with the user(s) via one or more user interfaces of the user device 200. The user interfaces include, but are not limited to: display/monitors illustrating graphical user interfaces; an audio system for providing audio instructions and receiving audio selections from a user; and/or input devices such as keyboards, mouse, and/or touchscreens for receiving inputs. While the chemical compound entry module 210 and the analysis request module 220 are shown as provided on two separate user devices 200, it should be understood that the chemical compound entry module 210 and the analysis request module 220 may be provided on the one user device 200.

In one form, the chemical compound system 100 includes a fragment identification module 110, a subgraph module 120, a node creation module 130, a node arrangement module 140, a node connection module 150, a node merging module 160, a chemical compound graph database 170, and a chemical analysis module 180. In one form, the user device 200-1 includes a chemical compound entry module 210 and the user device 200-2 includes an analysis request module 220. It should be readily understood that any one of the components of the chemical compound system 100 and/or the user devices 200 can be provided at the same location or distributed at different locations and communicably coupled accordingly.

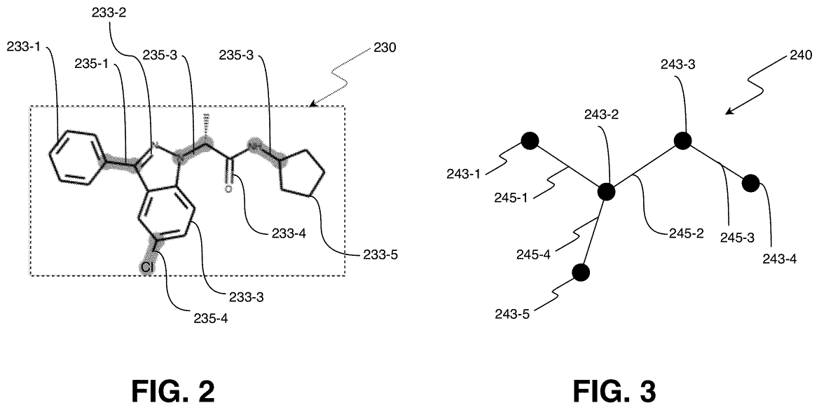

In one form, the fragment identification module 110 is configured to obtain information representing a given chemical compound provided by the chemical compound entry module 210. For example, and as shown in FIG. 2, a user (e.g., a developer of the chemical compound system 100) provides, using the chemical compound entry module 210, an image including a skeletal formula 230 representing 2-(5-Chloro-3-phenyl-1H-indazol-1-yl)-N-cyclopentylpropanamide (C₂₁H₂₂CIN₃O) to the fragment identification module 110. It should be understood that the user may provide other representations of the given chemical compound, such as text, voice commands corresponding to the chemical compound, etc., to the fragment identification module 110 and is not limited to the example described herein.

In response to receiving the skeletal formula 230, the fragment identification module 110 is configured to identify one or more fragments of the skeletal formula 230. In one form, the fragment identification module 110 identifies fragments connected to the ring molecules (e.g., monocycles, polycycles, etc.) of the skeletal formula 230. As an example and as shown in FIG. 2, the fragment identification module 110 identifies fragments 235-1, 235-2, 235-3, 235-4 (collectively referred to herein as fragments 235) as the fragments that are connected to ring molecules 233-1, 233-2, 233-3, 233-4, 233-5 (collectively referred to herein as ring molecules 233). It should be understood that the fragment identification module 110 may identify fragments that are not connected to the rings of the skeletal formula 230 in other forms, such as amide bonds.

With continuing reference to FIG. 1, the subgraph module 120 is configured to generate a subgraph based on the identified fragments 235. As an example and as shown in FIG. 3, the subgraph module 120 generates a subgraph 240 that includes ring molecule vertices 243-1, 243-2, 243-3, 243-4, 243-5 (collectively referred to herein as ring molecule vertices 243) that are connected by fragment edges 245-1, 245-2, 245-3, 245-4 (collectively referred to herein as fragment edges 245). In one form, the ring molecule vertices 243 correspond to the ring molecules 233, and the fragment edges 245 correspond to the identified fragments 235.

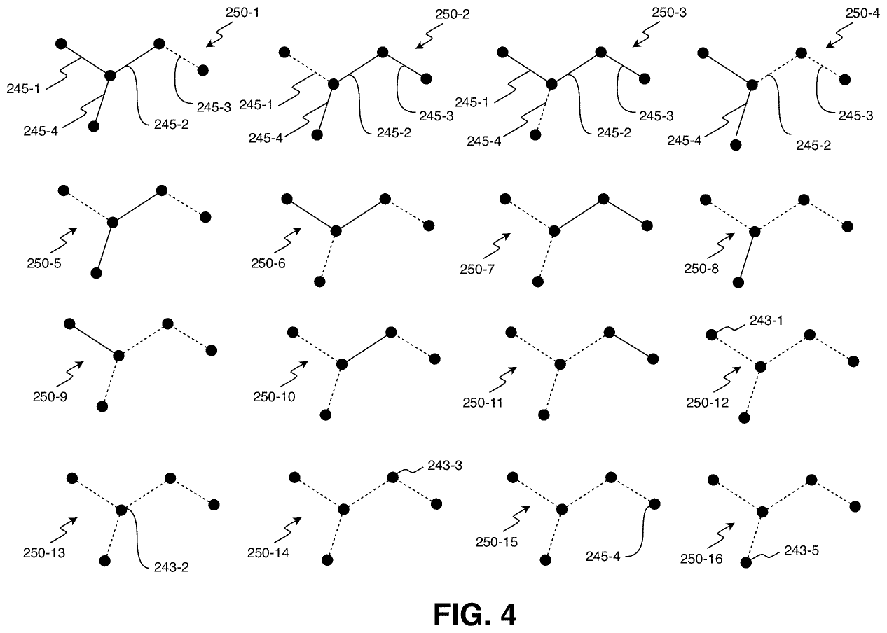

The subgraph module 120 is configured to generate a plurality of reduced subgraphs based on the subgraph 240. In one form, the subgraph module 120 generates the plurality of reduced subgraphs based on each substructure of the subgraph 240. As an example and as shown in FIG. 4, the subgraph module 120 generates: reduced subgraphs 250-1, 250-2, 250-3 that represent substructures having three of the fragment edges 245; reduced subgraphs 250-4, 250-5, 250-6, 250-7 that represent substructures having two of the fragment edges 245; reduced subgraphs 250-8, 250-9, 250-10, 250-11 that represent substructures having one of the fragment edges 245; and reduced subgraphs 250-12, 250-13, 250-14, 250-15, 250-16 that represent substructures having only one of the ring molecule vertices 243. The reduced subgraphs 250-1, 250-2, . . . 250-16 are collectively referred to herein as reduced subgraphs 250. While FIG. 4 illustrates the subgraph module 120 generating reduced subgraphs 250 for each substructure of the subgraph 240, it should be understood that the subgraph module 120 may only generate reduced subgraphs 250 representing structures that only have a predefined number of fragment edges 245 and/or predefined ring molecule vertices 243 in other forms.

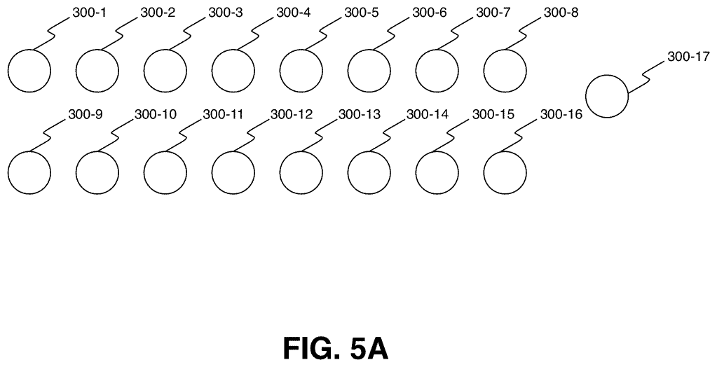

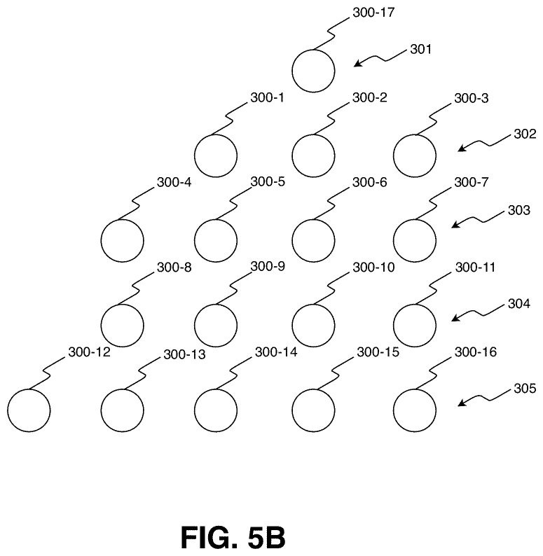

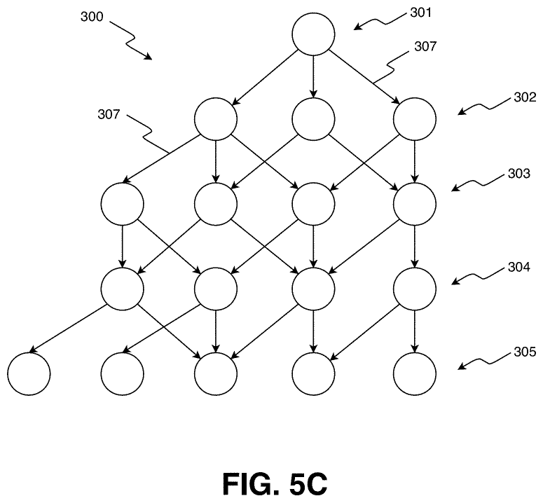

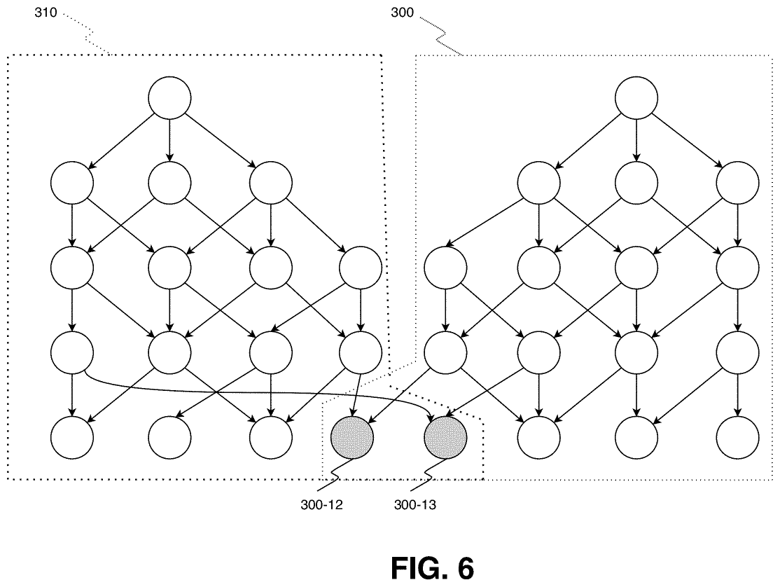

In one form, the node creation module 130 is configured to generate a plurality of nodes based on the subgraph 240 reduced subgraphs 250. In one form, the node creation module 130 is configured to generate a node for each of the reduced subgraphs 250 and the subgraph 240. As an example and as shown in FIG. 5A, the node creation module 130 is configured to generate node 300-17 that corresponds to subgraph 240 and nodes 300-1, 300-2, 300-3, 300-4, 300-5, 300-6, 300-7, 300-8, 300-9, 300-10, 300-11, 300-12, 300-13, 300-14, 300-15, 300-16 that each correspond to one of the reduced subgraphs 250. The nodes 300-1, 300-2, . . . , 300-17 are collectively referred to herein as nodes 300.

The node arrangement module 140 is configured to arrange the nodes 300 based on a number of fragment edges 245 of the subgraph 240 or respective reduced subgraph 250 associated with the given node 300. As an example and as shown in FIG. 5B, the nodes 300 are vertically arranged into a first row 301, a second row 302, a third row 303, a fourth row 304, and a fifth row 305. The first row 301 includes the node associated with the subgraph 240 (i.e., the node 300-17 associated with the subgraph 240, which includes n fragment edges 245). The second row 302 includes the nodes 300 associated with reduced subgraphs 250 having one less fragment edge than the first row 301 (i.e., the nodes 300-1, 300-2, 300-3 associated with reduced subgraphs 250-1, 250-2, 250-3, which each include n-1 fragment edges 245). The third row 303 includes the nodes 300 associated with reduced subgraphs 250 having one less fragment edge than the second row 302 (i.e., the nodes 300-4, 300-5, 300-6, 300-7 associated with reduced subgraphs 250-4, 250-5, 250-6, 250-7, which each include n-2 fragment edges 245). The fourth row 304 includes the nodes 300 associated with reduced subgraphs 250 having one less fragment edge than the third row 303 (i.e., the nodes 300-8, 300-9, 300-10, 300-11 associated with reduced subgraphs 250-8, 250-9, 250-10, 250-11, which each include n-3 fragment edges 245). The fifth row 305 includes the nodes 300 associated with reduced subgraphs 250 having one less fragment edge than the fourth row 304 (i.e., the nodes 300-12, 300-13, 300-14, 300-15, 300-16 associated with reduced subgraphs 250-12, 250-13, 250-14, 250-15, 250-16, which each include n-4 fragment edges 245).

The node connection module 150 is configured to connect the nodes 300 using edges 307 and based on one or more reduced graph rules. In one form, the one or more reduced graph rules include instructions for connecting the nodes 300 using edges 307 based on a nontransitive reduction routine. As an example and as shown in FIG. 5C, the node connection module 150 is configured to connect the nodes 300 using edges 307 to form a directed acyclic graph, such as a Hasse diagram. Furthermore, the reduced graph rules may include instructions for connecting the nodes 300 such that the edges 307 are nontransitive. As an example and as shown in FIG. 5C, none of the edges 307 connect nodes 300 that are also connected via a longer path (i.e., an alternate path with intermediate nodes 300). As a specific example of the nontransitive edge rule, none of the edges 307 connect node 300-17 (FIG. 5B) of the first row 301 to the nodes of the third, fourth, or fifth rows 303, 304, 305; none of the edges 307 connect the nodes 300 of the second row 302 to the nodes of the fourth or fifth rows 304, 305; and none of the edges 307 connect the nodes 300 of the to the nodes of the fifth row 305.

In one form, the node connection module 150 is configured to generate and store data entries based on the nodes 300 and the edges 307 in the chemical compound graph database 170. In one form, the subgraph 240 and the reduced subgraphs 250 correspond to the properties of the nodes 300 of the chemical compound graph database 170. As an example, the chemical compound graph database 170 may include various non-relational databases, such as a NoSQL database.

The node merging module 160 is configured to merge the nodes 300 with other nodes of the chemical compound graph database 170. In one form, the node merging module 160 identifies one or more shared nodes of the nodes 300 (i.e., nodes that are already stored in the chemical compound graph database 170 and associated with another chemical compound) and merges the nodes 300 at the one or more shared nodes. As an example and as shown in FIG. 6, the node merging module 160 may identify nodes 300-12, 300-13 as the shared nodes already stored in the chemical compound graph database 170 and associated with nodes 310 representing a second chemical compound. As such, the node merging module 160 may merge the nodes 300, 310 at the one or more shared nodes 300-12, 300-13.

In some forms, the node merging module 160 may repeat the merging routine described herein for each set of new nodes that are generated and stored in the chemical compound graph database 170 such that each shared node of the new nodes are merged with existing nodes and thus removing duplicate nodes. In some forms, the merged nodes generated by the node merging module 160 collectively form a nontransitive, directed acyclic graph/Hasse diagram (referred to herein as the semilattice structure) that defines the structural relationship between all of the molecules of the chemical compounds in the chemical compound graph database 170. The semilattice structure relates the molecular structures of the chemical compound graph database 170 and implicitly defines sequences of chemical transformations for navigating between any two molecules in the chemical compound graph database 170. Furthermore, the semilattice structure is independent of atom ordering by graph isomorphism, expresses a partial order relationship and a cover relationship (i.e., a child node of the semilattice structure is a proper substructure of its parent nodes, and vice versa), and obeys all the partial order properties of a join-semilattice (i.e., no two nodes have more than one parent in shared nor more than one child in shared).

In one form, the chemical analysis module 180 is configured to generate and/or provide a SAR analysis in response to a request for a SAR analysis of a given node via the analysis request module 220. In one form, the chemical analysis module 180 identifies a SAR neighborhood associated with the given node based on one or more SAR rules and in response to the request for the SAR analysis. In one form, the SAR rules provide instructions for identifying nodes of the semilattice structure that have similar chemical structures as the given node. In one form, the instructions include identifying the nodes include vertically traversing one or more levels of the semilattice structure along each available edge to identify one or more child nodes, grandchildren nodes, parent nodes, grandparent nodes, or a combination thereof.

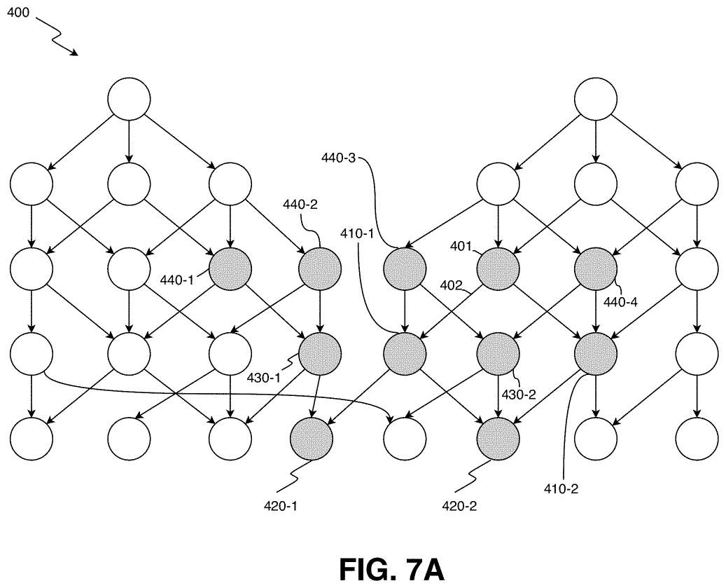

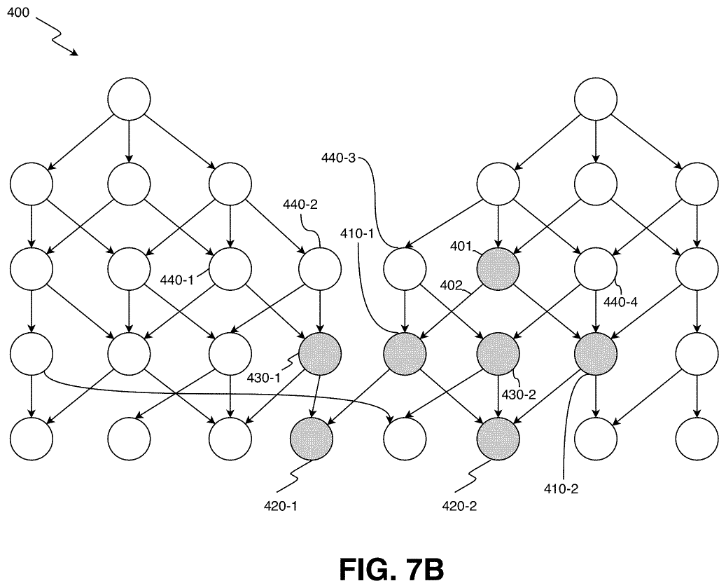

As an example and as shown in FIG. 7A, the user inputs a request for a SAR analysis of node 401 of semilattice structure 400. Based on the SAR rules, the chemical analysis module 180 may initially identify each child node and grandchildren node of the node 401 along each available edge, such as child nodes 410-1, 410-2 (collectively referred to as child nodes 410) and grandchildren nodes 420-1, 420-2 (collectively referred to as grandchildren nodes 420). Subsequently and based on the SAR rules, the chemical analysis module 180 may identify each parent node and grandparent node of the grandchildren nodes 420, such as parent nodes 430-1, 430-2 (collectively referred to as parent nodes 430) and grandparent nodes 440-1, 440-2, 440-3, 440-4 (collectively referred to as grandparent nodes 440). Accordingly, the chemical analysis module 180 may determine that the SAR neighborhood includes the node 401, the child nodes 410, the grandchildren nodes 420, the parent nodes 430, and the grandparent nodes 440. It should be understood that the SAR neighborhood may include any combination of the node 401, the child nodes 410, the grandchildren nodes 420, the parent nodes 430, and the grandparent nodes 440. As another example and as shown in FIG. 7B, the SAR neighborhood of node 401 does not include the grandparent nodes 440.

In response to identifying the SAR neighborhood, the chemical analysis module 180 is configured to perform a SAR analysis based on the nodes of the SAR neighborhood. As an example and referring to FIGS. 7A-7B, the chemical analysis module 180 may generate a Free-Wilson regression analysis, an additivity analysis, a non-additivity analysis based on the node 401, the child nodes 410, the grandchildren nodes 420, the parent nodes 430, and/or the grandparent nodes 440. As another example, the chemical analysis module 180 may provide the information corresponding to the node 401, the child nodes 410, the grandchildren nodes 420, the parent nodes 430, and/or the grandparent nodes 440 to the analysis request module 220, which in turn generates the SAR analysis. It should be understood that various other SAR analyses may be performed and are not limited to the examples described herein. Accordingly, by identifying the SAR neighborhood using the semilattice structure 400, relevant SAR analyses can be performed to verify that various modifications to a lead compound satisfy additivity, non-additivity, and/or other types of SAR thresholds/assumptions.

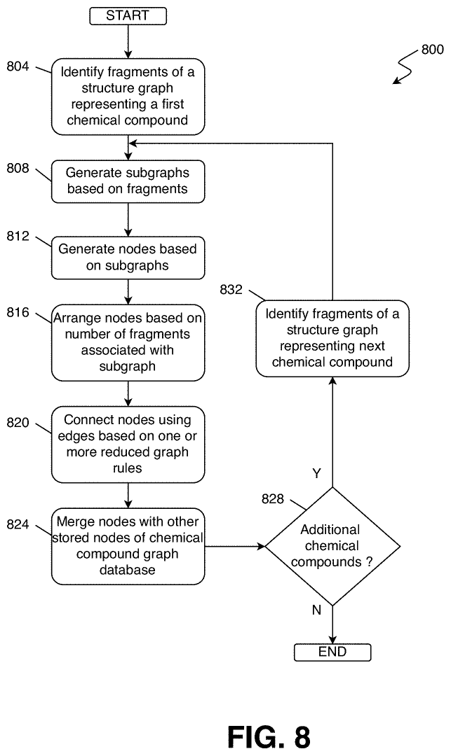

Referring to FIG. 8, a flowchart illustrating a routine 800 for generating the chemical compound graph database 170 of the chemical compound system 100 is shown. At 804, the chemical compound system 100 identifies fragments of a structure graph representing a first chemical compound. At 808, the chemical compound system 100 generates one or more subgraphs (e.g., the reduced subgraphs) based on the fragments of the first chemical compound. At 812, the chemical compound system 100 generates a plurality of nodes based on the subgraphs and arranges the nodes based on the number of fragments associated with each subgraph at 816.

At 820, the chemical compound system 100 connects the nodes using edges and based on one or more reduced graph rules. At 824, the chemical compound system 100 merges the nodes with other nodes stored in the chemical compound graph database 170. At 828, the chemical compound system 100 determines whether there are additional chemical compounds to be added to the chemical compound graph database 170. If so, the routine 800 proceeds to 832, where the chemical compound system 100 identifies fragments of a structure graph representing the next chemical compound and proceeds to 808. Otherwise, the routine 800 ends.

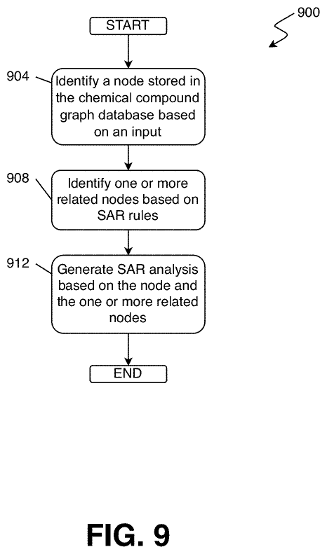

Referring to FIG. 9, a flowchart illustrating a routine 900 for obtaining a SAR analysis of a fragment of a given chemical compound of the chemical compound database 170 is shown. At 904, the chemical compound system 100 identifies a node stored in the chemical compound graph database 170 based on a user input received via the analysis request module 220. At 908, the chemical compound system 100 identifies one or more related nodes based on the one or more SAR rules. At 912, the chemical compound system 100 and/or the user device 200-2 generate the SAR analysis based on the node and the one or more related nodes, such as a Free-Wilson regression analysis, an additivity analysis, a non-additivity analysis.

Unless otherwise expressly indicated herein, all numerical values indicating mechanical/thermal properties, compositional percentages, dimensions and/or tolerances, or other characteristics are to be understood as modified by the word “about” or “approximately” in describing the scope of the present disclosure. This modification is desired for various reasons including industrial practice; material, manufacturing, and assembly tolerances; and testing capability.

As used herein, the phrase at least one of A, B, and C should be construed to mean a logical (A OR B OR C), using a non-exclusive logical OR, and should not be construed to mean “at least one of A, at least one of B, and at least one of C.”

The description of the disclosure is merely exemplary in nature and, thus, variations that do not depart from the substance of the disclosure are intended to be within the scope of the disclosure. Such variations are not to be regarded as a departure from the spirit and scope of the disclosure.

In the figures, the direction of an arrow, as indicated by the arrowhead, generally demonstrates the flow of information (such as data or instructions) that is of interest to the illustration. For example, when element A and element B exchange a variety of information, but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, the term module may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality, such as, but not limited to, transceivers, routers, input/output interface hardware, among others; or a combination of some or all of the above, such as in a system-on-chip.

The term memory is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general-purpose computer to execute one or more particular functions embodied in computer programs. The functional blocks, flowchart components, and other elements described above serve as software specifications, which can be translated into the computer programs by the routine work of a skilled technician or programmer.

What is claimed is:

1. A method for generating a chemical compound graph database, the method comprising:
identifying, using one or more processors configured to execute instructions stored in a nontransitory computer-readable medium, a first plurality of fragments of a first structure graph representing a first chemical compound;
generating, using the one or more processors, a first plurality of subgraphs of the first structure graph based on the first plurality of fragments;
generating, using the one or more processors, a first plurality of nodes based on the first plurality of subgraphs, wherein each node of the first plurality of nodes corresponds to a respective subgraph of the first plurality of subgraphs;
arranging, using the one or more processors, the first plurality of nodes based on a number of the first plurality of fragments associated with each of the first plurality of subgraphs; and
connecting, using the one or more processors, the first plurality of nodes using a first plurality of edges and based on one or more reduced graph rules.

2. The method of claim 1 further comprising:
identifying, using the one or more processors, a second plurality of fragments of a second structure graph representing a second chemical compound;
generating, using the one or more processors, a second plurality of subgraphs of the second structure graph based on the second plurality of fragments;
generating, using the one or more processors, a second plurality of nodes based on the second plurality of subgraphs, wherein each node of the second plurality of nodes corresponds to a respective subgraph of the second plurality of subgraphs;
arranging, using the one or more processors, the second plurality of nodes based on a number of the second plurality of fragments associated with each of the second plurality of subgraphs; and
connecting, using the one or more processors, the second plurality of nodes using a second plurality of edges and based on the one or more reduced graph rules.

3. The method of claim 2 further comprising:
identifying, using the one or more processors, one or more shared nodes from among the first plurality of nodes and the second plurality of nodes; and
merging, using the one or more processors, the first plurality of nodes and the second plurality of nodes at the one or more shared nodes.

4. The method of claim 3 further comprising generating one or more data entries of the chemical compound graph database based on the merged first plurality of nodes and the second plurality of nodes.

5. The method of claim 1, wherein each fragment of the first plurality of fragments is linked to a ring molecule of the first chemical compound.

6. The method of claim 1, wherein the one or more reduced graph rules further comprises connecting the first plurality of nodes using the first plurality of edges based on a nontransitive reduction routine.

7. The method of claim 1, wherein the one or more reduced graph rules further comprises connecting the first plurality of nodes using the first plurality of edges to form a Hasse diagram.

8. A system for generating a chemical compound graph database, the system comprising:
one or more processors; and
a nontransitory computer-readable medium comprising instructions that are executable by the one or more processors, wherein the instructions comprise:
identifying a first plurality of fragments of a first structure graph representing a first chemical compound;
generating a first plurality of subgraphs of the first structure graph based on the first plurality of fragments;
generating a first plurality of nodes based on the first plurality of subgraphs, wherein each node of the first plurality of nodes corresponds to a respective subgraph of the first plurality of subgraphs;
arranging the first plurality of nodes based on a number of the first plurality of fragments associated with each of the first plurality of subgraphs; and
connecting the first plurality of nodes using a first plurality of edges and based on one or more reduced graph rules.

9. The system of claim 8, wherein the instructions further comprise:
identifying a second plurality of fragments of a second structure graph representing a second chemical compound;
generating a second plurality of subgraphs of the second structure graph based on the second plurality of fragments;
generating a second plurality of nodes based on the second plurality of subgraphs, wherein each node of the second plurality of nodes corresponds to a respective subgraph of the second plurality of subgraphs;
arranging the second plurality of nodes based on a number of the second plurality of fragments associated with each of the second plurality of subgraphs; and
connecting the second plurality of nodes using a second plurality of edges and based on the one or more reduced graph rules.

10. The system of claim 9, wherein the instructions further comprise:
identifying one or more shared nodes from among the first plurality of nodes and the second plurality of nodes; and
merging the first plurality of nodes and the second plurality of nodes at the one or more shared nodes.

11. The system of claim 10, wherein the instructions further comprise generating one or more data entries of the chemical compound graph database based on the merged first plurality of nodes and the second plurality of nodes.

12. The system of claim 8, wherein each fragment of the first plurality of fragments is linked to a ring molecule of the first chemical compound.

13. The system of claim 8, wherein the one or more reduced graph rules further comprises connecting the first plurality of nodes using the first plurality of edges based on a nontransitive reduction routine.

14. The system of claim 8, wherein the one or more reduced graph rules further comprises connecting the first plurality of nodes using the first plurality of edges to form a Hasse diagram.

15. A method comprising:
identifying, using one or more processors configured to execute instructions stored in a nontransitory computer-readable medium, a node from among a plurality of nodes stored in a chemical compound graph database based on an input received by the one or more processors, wherein the node corresponds to one or more fragments of a chemical compound;
identifying, using the one or more processors, one or more related nodes from among the plurality of nodes associated with the node based on one or more structure-activity relationship rules; and
generating, using the one or processors, a structure activity relationship analysis based on the one or more related nodes and the node.

16. The method of claim 15, wherein the plurality of nodes form a Hasse diagram.

17. The method of claim 15, wherein the one or more structure-activity relationship rules comprise identifying, as the one or more related nodes, one or more child nodes associated with the node, one or more grandchildren nodes associated with the node, one or more parent nodes, one or more grandparent nodes, or a combination thereof.

18. The method of claim 15, wherein the structure activity relationship analysis includes a Free-Wilson regression analysis, an additivity analysis, a non-additivity analysis, or a combination thereof.

19. The method of claim 15, wherein:
the plurality of nodes include a first plurality of nodes and a second plurality of nodes;
the first plurality of nodes represent a first chemical compound;
the second plurality of nodes represent a second chemical compound;
the first plurality of nodes are connected by a first plurality of edges and based on a nontransitive reduction routine; and
the second plurality of nodes are connected by a second plurality of edges and based on the nontransitive reduction routine.

20. The method of claim 19, wherein the first plurality of nodes and the second plurality of nodes are merged at one or more shared nodes from among the first plurality of nodes and the second plurality of nodes.
